SEQUENCE LISTING (6 sequences)

SEQ ID NO: 1 - DNA, 121 bp, Homo sapiens
tcgattagcatgtaatgattttgacattggcagggtgataaaagggagaattgagagtatrgagggaagaaaataaatgcaaggagggagaaaaaagaggaaataaacaacaaaggaagga

SEQ ID NO: 2 - DNA, 121 bp, Homo sapiens
caaaccacccgtcgactgacaaatttagataagcaaatgtggtacggacacatagcgaaayattatctgaccataaaaaggagtggagtgctgataaacactacaacatggatagaccttg

SEQ ID NO: 3 - DNA, 121 bp, Homo sapiens
ttaaaattactttctaaagccaatcattctgcctaatacagggtcttcatttatttttagmtacctgaaactgagtctaaaaccacttctctctacttcctcttgtctttttcatttaaac

SEQ ID NO: 4 - DNA, 121 bp, Homo sapiens
gctgggctacctcctttgggagataggttctgccctgtcactgtctacaaaattgttaatrttcccaaagaaactgtctgggcccccaagccctcttttaagccaggaattgtgacatttt

SEQ ID NO: 5 - DNA, 121 bp, Homo sapiens
gggcaattcggctgtggatccaaatatgtacactccactcagcaaagtgaaactccaaagrcagccaaggtatttattacctgttgttaccagagcacatcccttggcgttttacacccca

SEQ ID NO: 6 - DNA, 121 bp, Homo sapiens
tgggccctctacagctgtcttggggctaaagggaagaagaggaaatgacacctctgctggyggaattatagcctgccagagttggaaaggacctcagagatgatcactcaagcccaccccc

(19) United States
(12) Patent Application Publication
(10) Pub. No.: US 2013/0236893 A1
(43) Pub. Date: Sep. 12, 2013

(54) METHOD OF DETECTING TYPE II DIABETES

(71) Applicants: Shiro Maeda, Yokohama-shi (JP); Takashi Kadowaki, Tokyo (JP); Toshimasa Yamauchi, Tokyo (JP); Kazuo Hara, Tokyo (JP)

(72) Inventors: Shiro Maeda, Yokohama-shi (JP); Takashi Kadowaki, Tokyo (JP); Toshimasa Yamauchi, Tokyo (JP); Kazuo Hara, Tokyo (JP)

(73) Assignees: TOKUSHUKAI, Tokyo (JP); RIKEN, Saitama (JP)

(21) Appl. No.: 13/819,821

(22) PCT Filed: Sep. 2, 2011
(86) PCT No.: PCT/JP2011/070536

§ 371 Date: Mar. 18, 2013

Related U.S. Application Data
(60) Provisional application No. 61/379,489, filed on Sep. 2, 2010.

(51) Int. Cl. C12Q 1/68

(52) U.S. Cl. CPC C12Q 1/6883
USPC 435/6.11; 536/24.31; 536/24.33

(57) ABSTRACT

A single-nucleotide polymorphism in the UBE2E2 locus or C2CD4A-C2CD4B locus is analyzed and type II diabetes is examined based on the results of the analysis.

### TECHNICAL FIELD

The present invention relates to a method of detecting type II diabetes (T2D).

### BACKGROUND OF THE INVENTION

T2D affects nearly 300 million individuals worldwide, and its escalating prevalence is a serious concern in many countries, including Japan. Although multiple genetic and environmental factors are thought to contribute to the pathogenesis of T2D, the precise mechanisms underlying the development and progression of the disease have not been fully elucidated.

Genome-wide association studies (GWAS) conducted in populations of European descent have identified 26 susceptibility loci for T2D at genome-wide significant levels. Recently, results of two GWAS in a Japanese population were simultaneously reported, however, their sample sizes were relatively small. One study was conducted using 82,343 SNP markers in stage 1 (187 individuals with T2D (cases) and 752 unaffected controls) (Nat. Genet. 40, 1092-1097 (2008)), and the other study was conducted using 207,097 SNP markers in stage 1 (194 cases and 1,558 controls) (Nat. Genet. 40, 1098-1102 (2008)). Both GWAS discovered the same T2D susceptibility locus (in KCNQ1); this result was also confirmed in east Asian and European populations (Nat. Genet. 40, 1092-1097 (2008) and Nat. Genet. 40, 1098-1102 (2008)). Although clinical features of T2D vary substantially across different population groups, population differences in genetic risk loci with genome-wide significant support remain poorly defined.

### DISCLOSURE OF THE INVENTION

An object of the present invention is to provide a method of detecting a risk of the onset of type II diabetes, or the presence or absence of the onset thereof.

The inventors of the present invention have intensively studied for solving the above-mentioned problems. As a result, the inventors of the present invention have found that single nucleotide polymorphisms in the UBE2E2 locus or C2CD4A-C2CD4B locus are associated with type II diabetes, thereby completed the present invention.

It is one aspect of the present invention is a method of detecting type II diabetes, comprising:

analyzing a single-nucleotide polymorphism in the UBE2E2 locus or C2CD4A-C2CD4B locus, and

detecting type II diabetes based on the result of the analysis.

It is another aspect of the present invention is the method as described above, wherein a single-nucleotide polymorphism in the UBE2E2 locus is a polymorphism of a nucleotide corresponding to the nucleotide at position 61 of SEQ ID NO: 1, SEQ ID NO: 2, or SEQ ID NO: 3, or of a nucleotide in linkage disequilibrium with the nucleotide.

It is another aspect of the present invention is the method as described above, wherein a single-nucleotide polymorphism in the C2CD4A-C2CD4B locus is a polymorphism of a nucleotide corresponding to the nucleotide at position 61 of SEQ ID NO: 4, SEQ ID NO: 5, or SEQ ID NO: 6, or of a nucleotide in linkage disequilibrium with the nucleotide.

It is another aspect of the present invention is a probe for detecting type H diabetes, which comprises a sequence of 10 or more consecutive nucleotides in SEQ ID NO: 1, 2, 3, 4, 5, or 6 including the nucleotide at position 61, or a complementary sequence thereof.

It is another aspect of the present invention is a primer for detecting type II diabetes, which is capable of amplifying a region comprising the nucleotide at position 61 of SEQ ID NO: 1, 2, 3, 4, 5, or 6.

### BRIEF DESCRIPTION OF THE DRAWING

FIG. 1 is a scheme for GWAS.

### DESCRIPTION OF THE PREFERRED EMBODIMENTS

### <1> Detection Method of the Present Invention

The method of the present invention comprises analyzing a single nucleotide polymorphism associated with type II diabetes in the UBE2E2 locus or C2CD4A-C2CD4B locus, and detecting the type II diabetes based on the analytical result. In the present invention, the term “detection” includes detection of a risk of the onset of type II diabetes and detection of the presence or absence of the onset.

As the UBE2E2 locus, UBE2E2 locus on human chromosome 3 is preferable. For example, it may be a locus comprising a sequence registered as Accession No. NT_{—}022517.18 in the database of the National Center for Biotechnology Information (NCBI).

As the C2CD4A-C2CD4B locus, C2CD4A-C2CD4B locus on human chromosome 15 is preferable. For example, it may be a locus comprising a sequence registered as Accession No. NT_{—}010194.17 in the database of NCBI.

In addition, UBE2E2 locus and C2CD4A-C2CD4B locus are not limited to the genes comprising the above-mentioned sequences because there are racial differences and so on in these genes and substitutions, deletions, or the like may occur in nucleotides other than those associated with type II diabetes.

The exemplary information about UBE2E2 is shown below.
UBE2E2 ubiquitin-conjugating enzyme E2E 2 (UBC4/5 homolog, yeast) [Homo sapiens]

### Genomic NT_{—}022517.18 23184783.23572295

mRNA NP_{—}689866.1
The C2CD4A-C2CD4B locus comprises C2CD4A and C2CD4B.
The exemplary information about C2CD4A is shown below.
C2CD4A C2 calcium-dependent domain containing 4A [Homo sapiens]

### Genomic NT_{—}010194.17 33149732.33153672

mRNA NP_{—}997205.2
The exemplary information about C2CD4B is shown below.
C2CD4B C2 calcium-dependent domain containing 4B [Homo sapiens]
Genomic NT_{—}010194.17 33246293.33248038, complement
mRNA NP_{—}001007596.2

Single nucleotide polymorphisms in the UBE2E2 locus associated with type II diabetes are not particularly limited, and examples thereof include a polymorphism of a nucleotide corresponding to the nucleotide at position 61 of SEQ ID NO: 1 (rs6780569), a polymorphism at position 61 of SEQ ID NO: 2 (rs9812056), and a polymorphism at position 61 of SEQ ID NO: 3 (rs7612463).

rs6780569 NT_022517.18:g.23138484
G (risk allele) > A
SEQ ID NO: 1
TCGATTAGCA TGTAATGATT TTGACATTGG CAGGGTGATA
AAAGGGAGAA TTGAGAGTAT
R
GAGGGAAGAA AATAAATGCA AGGAGGGAGA AAAAAGAGGA
AATAAACAAC AAAGGAAGGA
rs9812056 NT_022517.18:g.23144024
T (risk allele) > C
SEQ ID NO: 2
CAAACCACCC GTCGACTGAC AAATTTAGAT AAGCAAATGT
GGTACGGACA CATAGCGAAA
Y
ATTATCTGAC CATAAAAAGG AGTGGAGTGC TGATAAACAC
TACAACATGG ATAGACCTTG
rs7612463 NT_022517.18:g.23276450
C (risk allele) > A
SEQ ID NO: 3
TTAAAATTAC TTTCTAAAGC CAATCATTCT GCCTAATACA
GGGTCTTCAT TTATTTTTAG
M
TACCTGAAAC TGAGTCTAAA ACCACTTCTC TCTACTTCCT
CTTGTCTTTT TCATTTAAAC

Single nucleotide polymorphisms in the C2CD4A-C2CD4B locus associated with type II diabetes are not particularly limited, and examples thereof include a polymorphism of a nucleotide corresponding to the nucleotide at position 61 of SEQ ID NO: 4 (rs7172432), a polymorphism at position 61 of SEQ ID NO: 5 (rs1436953), and a polymorphism at position 61 of SEQ ID NO: 6 (rs1370176).

rs7172432 NT_010194.17:g.33186946
A (risk allele) > G
SEQ ID NO: 4
GCTGGGCTAC CTCCTTTGGG AGATAGGTTC TGCCCTGTCA
CTGTCTACAA AATTGTTAAT
R
TTCCCAAAGA AACTGTCTGG GCCCCCAAGC CCTCTTTTAA
GCCAGGAATT GTGACATTTT
rs1436953 NT_010194.17:g.33204571
C (risk allele) > T
SEQ ID NO: 5
GGGCAATTCG GCTGTGGATC CAAATATGTA CACTCCACTC
AGCAAAGTGA AACTCCAAAG
R
CAGCCAAGGT ATTTATTACC TGTTGTTACC AGAGCACATC
CCTTGGCGTT TTACACCCCA

In SEQ ID NO: 5, the sequence of the antisense strand is shown so that SNP is shown as R (G (risk allele)>A).

rs1370176 NT_010194.17:g.33187791
G (risk allele) > A
SEQ ID NO: 6
TGGGCCCTCT ACAGCTGTCT TGGGGCTAAA GGGAAGAAGA
GGAAATGACA CCTCTGCTGG
Y
GGAATTATAG CCTGCCAGAG TTGGAAAGGA CCTCAGAGAT
GATCACTCAA GCCCACCCCC

In SEQ ID NO: 6, the sequence of the antisense strand is shown so that SNP is shown as Y (C (risk allele)>T).

The phrase “correspond to” means a corresponding nucleotide in a region containing the above-mentioned sequence on the human UBE2E2 locus or C2CD4A-C2CD4B locus. Even if the above-mentioned sequence is slightly modified at a position other than the SNP depending on a racial difference or the like, an analysis of the corresponding nucleotide therein may also be included.

The type II diabetes can be detected by analyzing the above-mentioned nucleotide polymorphisms singly or in combination. In addition, type II diabetes may be detected with respect to a polymorphism which is in linkage disequilibrium (r²>0.5, preferably r²>0.8) with the above-mentioned single nucleotide polymorphisms.

The sequence in the UBE2E2 locus or C2CD4A-C2CD4B locus may be analyzed with respect to either of its sense strand or antisense strand.

Samples to be used in analysis of genetic polymorphisms in UBE2E2 locus or C2CD4A-C2CD4B locus include, but not limited to, body fluid such as urine and blood, cells such as mucous cells, and body hair such as scalp hair. For the analysis of genetic polymorphisms, these samples may be directly used, but preferably chromosomal DNA is isolated from these samples by ordinary methods and then used for the analysis.

The analysis of genetic polymorphisms in UBE2E2 locus or C2CD4A-C2CD4B locus can be performed by conventional techniques for analyzing the genetic polymorphisms. Examples of the analysis include, but not limited to, sequence analysis, PCR, and hybridization.

The sequencing can be performed by conventional procedures. Specifically, a sequencing reaction is performed using a primer located several tens of nucleotides 5′ side from a polymorphic site. From the result of such an analysis, the kind of the nucleotide on the corresponding position can be determined. Preferably, when the sequencing is carried out, a fragment containing a polymorphic nucleotide is amplified by PCR or the like.

Further, the analysis can be carried out by detecting the presence of an amplified product in PCR. For instance, primers having a sequence corresponding to a region containing a polymorphic site and corresponding to the respective polymorphic nucleotides are prepared and then used in PCR, followed by detecting the presence of an amplified product to determine the kind of the polymorphic nucleotide.

Alternatively, the presence of an amplified product may be determined using a LAMP method (JP 3313358 B), a nucleic acid sequence-based amplification method (NASBA method; JP 2843586 B), and an ICAN method (JP 2002-233379 A). Any of other methods, such as a single-chain amplification method, may also be employed.

Further, a DNA fragment containing the polymorphic site may be amplified and the amplified product may be then electrophoresed, followed by determining the kind of the nucleotide based on a difference in mobility. An example of such a method includes single-strand conformation polymorphism (PCR-SSCP) (Genomics. 1992 Jan. 1; 12(1): 139-146). Specifically, at first, a DNA containing a polymorphic site in UBE2E2 locus or C2CD4A-C2CD4B locus is amplified and the amplified DNA is then dissociated to single stranded DNAs. Subsequently, the dissociated single stranded DNAs are separated on a non-denaturing gel and the kind of the nucleotide can be then determined based on a difference in mobilities of the dissociated single stranded DNAs on the gel.

Further, when a polymorphic nucleotide is included in a restriction-enzyme recognition sequence, the analysis may depend on the presence or absence of digestion with a restriction enzyme (RFLP method). In this case, at first, a DNA sample is digested with a restriction enzyme. The DNA fragment is then separated, thereby allowing the determination of the kind of the nucleotide based on the size of the detected DNA fragment.

Based on the polymorphism analyzed by the method as described above, type II diabetes can be detected.

For instance, in the case of detecting type II diabetes on the basis of a nucleotide corresponding to the nucleotide at position 61 of SEQ ID NO: 1 of the UBE2E2 locus, when the nucleotide is G, it is indicated that a risk of the onset of type II diabetes is high, or a possibility of suffering from type II diabetes is high. In addition, type II diabetes may be detected by considering a polymorphism of an allelic gene. For example, when the genotype is GG or AG allele, it can be indicated that a risk of the onset of type II diabetes is higher, or a possibility of suffering from type II diabetes is higher, as compared with AA allele.

In the case of detecting type II diabetes on the basis of a nucleotide corresponding to the nucleotide at position 61 of SEQ ID NO: 2 of the UBE2E2 locus, when the nucleotide is T, it is indicated that a risk of the onset of type II diabetes is high, or a possibility of suffering from type II diabetes is high. In addition, type II diabetes may be detected by considering a polymorphism of an allelic gene. For example, when the genotype is TT or TC allele, it can be indicated that a risk of the onset of type II diabetes is higher, or a possibility of suffering from type II diabetes is higher, as compared with CC allele.

In the case of detecting type II diabetes on the basis of a nucleotide corresponding to the nucleotide at position 61 of SEQ ID NO: 3 of the UBE2E2 locus, when the nucleotide is C, it is indicated that a risk of the onset of type II diabetes is high, or a possibility of suffering from type II diabetes is high. In addition, type II diabetes may be detected by considering a polymorphism of an allelic gene. For example, when the genotype is CC or CA allele, it is indicated that a risk of the onset of type II diabetes is higher, or a possibility of suffering from type II diabetes is higher, as compared with AA allele.

In the case of detecting type II diabetes on the basis of a nucleotide corresponding to the nucleotide at position 61 of SEQ ID NO: 4 of the C2CD4A-C2CD4B locus, when the nucleotide is A, it is indicated that a risk of the onset of type II diabetes is high, or a possibility of suffering from type II diabetes is high. In addition, type II diabetes may be detected by considering a polymorphism of an allelic gene. For example, when the genotype is AA or AG allele, it is indicated that a risk of the onset of type II diabetes is higher, or a possibility of suffering from type II diabetes is higher, as compared with GG allele.

In the case of detecting type II diabetes on the basis of a nucleotide corresponding to the nucleotide at position 61 of SEQ ID NO: 5 of the C2CD4A-C2CD4B locus, when the nucleotide is G, it is indicated that a risk of the onset of type II diabetes is high, or a possibility of suffering from type II diabetes is high. In addition, type II diabetes may be detected by considering a polymorphism of an allelic gene. For example, when the genotype is GG or GA allele, it is indicated that a risk of the onset of type II diabetes is higher, or a possibility of suffering from type II diabetes is higher, as compared with AA allele.

In the case of detecting type II diabetes on the basis of a nucleotide corresponding to the nucleotide at position 61 of SEQ ID NO: 6 of the C2CD4A-C2CD4B locus, when the nucleotide is C, it is indicated that a risk of the onset of type II diabetes is high, or a possibility of suffering from type II diabetes is high. In addition, type II diabetes may be detected by considering a polymorphism of an allelic gene. For example, when the genotype is CC or CT allele, it is indicated that a risk of the onset of type II diabetes is higher, or a possibility of suffering from type II diabetes is higher, as compared with TT allele.

### <2> Detection Agent of the Present Invention

In the present invention, detection agents, such as primers and probes, for detecting type II diabetes are provided. Examples of the probes include: a probe comprising a consecutive sequence in SEQ ID NO: 1, 2, 3, 4, 5, or 6 including the nucleotide at position 61 or a complementary sequence thereof.

Further, examples of the primers include: a primer capable of distinguishing a polymorphism of the nucleotide at position 61 of SEQ ID NO: 1, 2, 3, 4, 5, or 6, for example, a primer capable of amplifying a region comprising the nucleotide at position 61 of SEQ ID NO: 1, 2, 3, 4, 5, or 6. Primers may be a primer set of a forward primer and a reverse primer designed on both sides of a region (preferably region having a length of 50 to 1,000 nucleotides) containing the polymorphic site. In addition, when used in a sequence analysis or a single chain amplification, an example of the primer may be one having a 5′-side region from the above-mentioned polymorphic nucleotides, preferably having a sequence of the region 30 to 100 nucleotide upstream from the polymorphic site, or one having a sequence complementary to 3′-side region from the above-mentioned polymorphic nucleotides, preferably having a sequence complementary to the region 30 to 100 nucleotide downstream from the polymorphic site. The primers to be used for determining the polymorphisms on the basis of the presence or absence of the amplification in PCR include a primer comprising a consecutive sequence in SEQ ID NO: 1, 2, 3, 4, 5, or 6 including the above-mentioned polymorphic nucleotide on the 3′-side and a primer comprising a sequence complementary to the consecutive sequence in SEQ ID NO: 1, 2, 3, 4, 5, or 6 including the above-mentioned polymorphic nucleotide and containing a nucleotide complementary to the polymorphic nucleotide on the 3′-side.

The length of such primers and probes is not particularly limited, for instance, oligonucleotides with a length of 10 to 100 nucleotides are preferable, oligonucleotides with a length of 15 to 50 nucleotides are more preferable and oligonucleotides with a length of 20 to 35 nucleotides are more preferable. In addition, the detection agents of the present invention may further comprise PCR polymerase and buffer as well as these primers and probes.

Another method of detecting type II diabetes comprises analyzing an expression level (mRNA or protein) of UBE2E2 or C2CD4A and/or C2CD4B and detecting type II diabetes based on the result of the analysis. If the expression level is altered in a test subject as compared to a control subject without type II diabetes, it is indicated that the subject has a higher risk of the onset of type II diabetes, or has a possibility of suffering from type II diabetes. Here, the meaning of the term “altered expression” includes decreased expression as well as enhanced expression.

### <3> Screening Method

The screening method of the present invention is a method for screening a remedy for type II diabetes, comprising the steps of: adding a pharmaceutical candidate substance to a screening system comprising UBE2E2 or C2CD4A and/or C2CD4B measuring the activity of UBE2E2 or C2CD4A and/or C2CD4B; and selecting a substance that alters the activity.

The another screening method of the present invention is a method for screening a remedy for type II diabetes, comprising the steps of: adding a pharmaceutical candidate substance to a screening system such as cultured cell which expresses UBE2E2 or C2CD4A and/or C2CD4B; measuring the expression level (mRNA or protein) of UBE2E2 or C2CD4A and/or C2CD4B; and selecting a substance that alters the expression level.

The pharmaceutical candidate substance is not particularly limited, and may be a low-molecular synthetic compound or a compound derived from a natural source. Further, it may be a peptide. Individual test substances or a compound library comprising these substances may be used in screening. Among these candidate substances, a substance that alters the activity or expression level of UBE2E2 or C2CD4A and/or C2CD4B is selected as a therapeutic drug for type II diabetes. Here, the meaning of the term “alter” includes decreasing the activity (or expression) as well as enhancing the activity (or expression level).

### EXAMPLES

The present invention is explained by Examples below, but the scope of the invention is not limited thereto.

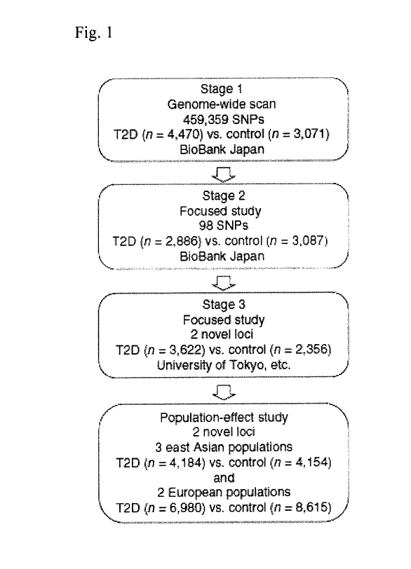

We conducted a GWAS for T2D in a Japanese population with a three-stage study design and performed follow-up studies in additional populations (FIG. 1). We first genotyped 4,878 individuals with T2D (this group is termed here case 1) and 3,345 controls (termed here control 1) collected from BioBank Japan (//biobankjp.org/) using the Illumina HumanHap610-Quad and 550K BeadChip, respectively. We first performed principal component analysis and identified two main clusters for our Japanese population, Hondo and Ryukyu, as reported previously (Am. J. Hum. Genet. 83, 445-456 (2008)). We then selected 7,541 subjects belonging to the Hondo cluster (4,470 cases and 3,071 controls) for an association study with T2D in the stage 1 genome scan (FIG. 1). We compared the genotype frequencies of 459,359 successfully genotyped SNPs using the Armitage test for trend with an additive association model (genomic inflation score λ=1.10, referenced λ_{1,000}=1.03) (Nat. Genet. 36, 388-393 (2004)) and identified one SNP within KCNQ1 that showed association at genome-wide significance16 (P<5×10⁻⁸).

We further examined the 100 SNPs showing the smallest P values in the stage 2 analysis, which were derived from 61 distinct loci, and we attempted to genotype these 100 SNPs in 2,886 individuals with T2D (termed case 2) and 3,087 controls (termed control 2) (FIG. 1). We successfully obtained data for 98 SNPs, and our combined analysis revealed that 20 of these SNPs had genome-wide significant association16 with T2D (P<5×10⁻⁸). Among them, 18 SNPs mapped to KCNQ1, CDKAL1, CDKN2B and TCF7L2. The KCNQ1 and CDKAL1 loci had already been reported to have genome-wide significant association with T2D in both European and Japanese populations. Moreover, we identified two previously unreported SNPs located in UBE2E2 on chromosome 3 that have modest effect sizes (odds ratio (OR)=1.21) and higher risk allele frequencies compared to the KCNQ1 SNPs in the Japanese population analyzed here (Table 1). We found three additional SNPs with borderline association (defined as P<1×10⁻⁷), including one additional SNP in UBE2E2 and two SNPs in C2CD4A-C2CD4B on chromosome 15.

We then focused on these two previously unreported loci (in UBE2E2 and C2CD4A-C2CD4B) for further analysis. Among the four SNPs within the UBE2E2 locus, rs6780569 and rs9812056 were in absolute linkage disequilibrium (LD) (r²=1), whereas the other SNPs were in moderate LD each other (r²=0.10-0.48). The three SNPs in the C2CD4A-C2CD4B locus (rs7172432, rs1436953 and rs1370176) were in high LD with each other (r²=0.62-0.80).

To validate the association of these two new loci, we genotyped a third set of Japanese cases and controls (stage 3, 3,622 T2D cases and 2,356 controls) (FIG. 1). The results indicated that the addition of the stage 3 results in the meta-analyses of the Japanese populations further strengthened the original association of these loci with T2D (UBE2E2: rs6780569, P=4.37×10⁻⁹; rs7612463, P=2.27×10⁻⁹; rs9812056, P=1.83×10⁻⁸; Table 2; C2CD4A-C2CD4B: rs7172432, P=3.66×10⁻⁹; rs1436953, P=2.19×10⁻⁸; Table 3). Moreover, the association of the SNPs in both loci remained genome-wide significant in the meta-analysis of the three Japanese populations when we used P values corrected with the genomic inflation score (λ=1.10) found in the initial GWAS (UBE2E2: rs7612463, P=2.10×10⁻⁸; C2CD4A-C2CD4B: rs7172432, P=4.88×10⁻⁸).

We further examined both loci in three east Asian populations (4,184 T2D cases and 4,154 controls) and two European populations (6,980 T2D cases and 8,615 controls) (FIG. 1). The association of both loci was replicated in these three east Asian populations (rs7612463 at UBE2E2, P=3.06×10⁻², Table 2; rs7172432 at C2CD4A-C2CD4B, P=1.26×10⁻²; Table 3), and integration of all results for the three Japanese and three east Asian populations further strengthened the association of these loci with T2D (rs7612463 in UBE2E2, P=9.16×10⁻¹⁰, OR=1.15, 95% CI 1.10-1.21; rs7172432 in C2CD4A-C2CD4B, P=2.61×10⁻¹⁰, OR=1.12, 95% CI 1.08-1.16). In the European populations, we replicated the association of C2CD4A-C2CD4B (rs7172432, P=6.36×10⁻⁵), and a combined analysis of all populations gave P=8.78×10⁻¹⁴. We failed to observe a significant association of SNPs in UBE2E2 with T2D in the European populations (P>0.05; Table 2).

TABLE 1
SNPs associated with T2D in the Japanese population

| | | | | Stage 1 | | | Stage 2 | | | | |
|---|---|---|---|---|---|---|---|---|---|---|---|
| | | Nearest | Risk | RAF | RAF | | RAF | RAF | | | |
| SNP, alteration | Chr. | gene | allele | (cases) | (controls) | P [symbol] ^{a} | (cases) | (controls) | P_{add} | Combined P | OR (95% CI) |
| rs2237892, C > T | 11 | KCNQ1 | C | 0.660 | 0.614 | 5.03 × 10⁻⁸ | 0.669 | 0.611 | 7.41 × 10⁻⁸ | 6.66 × 10⁻¹⁶ | 1.25 (1.19-1.31) |
| | | | | | | (1.07 × 10⁻⁸) | | | | | |
| rs2206734, C > T | 6 | CDKAL1 | T | 0.449 | 0.407 | 1.45 × 10⁻⁶ | 0.453 | 0.405 | 1.93 × 10⁻⁷ | 1.86 × 10⁻¹³ | 1.20 (1.14-1.26) |
| | | | | | | (4.28 × 10⁻⁷) | | | | | |
| rs2383208, A > G | 9 | CDKN2B | A | 0.615 | 0.584 | 3.80 × 10⁻⁴ | 0.624 | 0.570 | 3.15 × 10⁻⁹ | 1.45 × 10⁻¹¹ | 1.19 (1.13-1.24) |
| | | | | | | (1.92 × 10⁻⁴) | | | | | |
| rs7901695, T > C | 10 | TCF7L2 | C | 0.056 | 0.040 | 1.23 × 10⁻⁵ | 0.056 | 0.042 | 2.29 × 10⁻⁴ | 4.53 × 10⁻⁹ | 1.41 (1.26-1.58) |
| | | | | | | (4.49 × 10⁻⁶) | | | | | |
| rs6780569, G > A | 3 | UBE2E2 | G | 0.850 | 0.822 | 1.08 × 10⁻⁵ | 0.856 | 0.833 | 4.61 × 10⁻⁴ | 6.76 × 10⁻⁹ | 1.21 (1.14-1.30) |
| | | | | | | (3.90 × 10⁻⁶) | | | | | |
| rs1470579, A > C | 3 | IGF2BP2 | C | 0.365 | 0.330 | 3.52 × 10⁻⁵ | 0.367 | 0.338 | 9.19 × 10⁻⁴ | 5.20 × 10⁻⁸ | 1.15 (1.09-1.21) |
| | | | | | | (1.42 × 10⁻⁵) | | | | | |
| rs7172432, A > G | 15 | C2CD4A- | A | 0.598 | 0.559 | 9.43 × 10⁻⁶ | 0.591 | 0.564 | 3.79 × 10⁻³ | 7.48 × 10⁻⁸ | 1.14 (1.09-1.20) |
| | | C2CD4B | | | | (3.35 × 10⁻⁶) | | | | | |

The top SNP at each locus is shown. Chr., chromosome; RAF, risk allele frequency: OR. odds ratio.
^{a}P values corrected for genomic control are presented and the uncorrected P values are in parentheses. [symbol]
indicates data missing or illegible when filed

TABLE 2
Association of SNPs in the UBE2E2 locus with T2D

| | | | | | | P for |
|---|---|---|---|---|---|---|
| | n (T2D/CN) | RAF (cases) | RAF (controls) | P | OR (95% CI) | heterogeneity |
| rs6780569 | | | | | | |
| First set (Japanese 1) | 4.338/3.071 | 0.849 | 0.822 | 1.10 × 10⁻⁵ | 1.22 (1.12-1.33) | |
| Second set (Japanese 2) | 2,886/3.073 | 0.856 | 0.833 | 4.61 × 10⁻⁴ | 1.19 (1.08-1.32) | |
| Third set (Japanese 3) | 3.571/2.309 | 0.846 | 0.832 | 0.0357 | 1.11 (1.01-1.23) | |
| All Japanese^{a} | 10.795/8.453 | 0.850 | 0.828 | 4.37 × 10⁻⁹ | 1.18 (1.12-1.25) | 0.397 |
| East Asian without Japanese | 2.010/1.945 | 0.825^{b} | 0.809^{b} | 0.0565 | 1.16 (1.00-1.25) | — |
| All east Asian^{a} | 12.805/10.398 | 0.846 | 0.825 | 1.04 × 10⁻⁹ | 1.17 (1.11-1.23) | 0.463 |
| All European^{a} | 3.551/4.882 | 0.898^{c} | 0.898^{c} | 0.976 | 1.00 (0.91-1.11) | — |
| All populations^{a} | 16.356/15.280 | | | | | 0.0469 |
| rs7612463 | | | | | | |
| First set (Japanese 1) | 4.338/3.071 | 0.849 | 0.825 | 1.03 × 10⁻⁴ | 1.19 (1.09-1.30) | |
| Second set (Japanese 2) | 2.613/3.073 | 0.860 | 0.835 | 1.76 × 10⁻⁴ | 1.22 (1.10.-1.35) | |
| Third set (Japanese 3) | 3.492/2.244 | 0.856 | 0.838 | 8.16 × 10⁻³ | 1.15 (1.04-1.28) | |
| All Japanese^{a} | 10.443/8.388 | 0.854 | 0.832 | 2.27 × 10⁻⁹ | 1.19 (1.12-1.26) | 0.754 |
| East Asian without Japanese^{a} | 4.143/4.062 | 0.825^{b} | 0.811^{b} | 0.0306 | 1.09 (1.01-1.18) | 0.881 |
| All east Asian^{a} | 14.586/12.450 | 0.846 | 0.825 | 9.16 × 10⁻¹⁰ | 1.15 (1.10-1.21) | 0.597 |
| All European^{a} | 6.476/8.441 | 0.886^{c} | 0.884^{c} | 0.708 | 1.01 (0.94-1.09) | 0.724 |
| All populations^{a} | 21.062/20.891 | | | | | 0.0804 |
| rs9812056 | | | | | | |
| First set (Japanese 1) | 4.338/3.071 | 0.850 | 0.825 | 3.99 × 10⁻⁵ | 1.21 (1.10-1.32) | |
| Second set (Japanese 2) | 2.883/3.071 | 0.857 | 0.836 | 1.14 × 10⁻³ | 1.18 (1.07-1,31) | |
| Third set (Japanese 3) | 3.587/2.318 | 0.848 | 0.833 | 0.0253 | 1.12 (1.02-1.24) | |
| All Japanese^{a} | 10.808/8.460 | 0.851 | 0.831 | 1.83 × 10-8 | 1.17 (1.11-1.24) | 0.572 |
| East Asian without Japanese^{a} | 2.765/2.561 | 0.835^{b} | 0.819^{b} | 0.0268 | 1.12 (1.01-1.24) | 0.840 |
| All east Asian^{a} | 13.573/11.021 | 0.848 | 0.828 | 2.01 × 10⁻⁹ | 1.16 (1.11-1.22) | 0.782 |
| AM European | 3.229/3.540 | 0.898^{c} | 0.904^{c} | 0.245 | 0.94 (0.84-1.05) | — |
| All populations^{a} | 16.802/14.561 | | | | | 0.0189 |

^{a}Combined analysis with [symbol] [symbol] test using a fixed effect model; the weighted means of the risk allele frequencies are presented.
^{b}Weighted means for risk allele frequencies for three east Asian populations (Singaporean Han Chinese/Hong Kong Han Chinese/Korean populations): rs6780569. T2D 0.825/-/-, CN 0.809/-/-: rs7612463, T2D 0.827/0.829/0.819. CN 0.813/0.810/0.8 [symbol] 9 [symbol] rs9812056, T2D 0.835/-/0.836, CN 0.819/-/0.818. Singaporean Han Chinese popuiation. n = 2.010 T2D cases and 1,945 CN cases [symbol] Hong Kong Han Chinese population, n = 1,416 T2D cases and 1,577 CN cases: Korean population, n = 758 T2D cases and 632 CN cases.
^{c}Weighted means for risk allele frequencies tor two European populations (Danish and French populations): rs6780569. T2D 0 898/-, CN 0.898/- [symbol] rs7612463. T2D 0.882/0.891 CN. 0.882/0.888: rs9812056. T2D -/0.898, CN -/0.904. Danser popaiation, n = 3,692 T2D cases and 5.045 CN cases: French population. n = 3.288 T2D cases and 3,569 CN cases. CN. [symbol]
indicates data missing or illegible when filed

TABLE 3
Association of SNPs within the C2CD4A-C2CD4B locus with T2D

| | | | | | | P for |
|---|---|---|---|---|---|---|
| | n (T2D/CN) | RAF (cases) | RAF (controls) | P | OR (95% CI) | heterogeneity |
| rs7172432 | | | | | | |
| First set (Japanese 1) | 4.337/3.070^{a} | 0.597 | 0.559 | 3.77 × 10⁻⁶ | 1.17 (1.09-1.25) | |
| Second set (Japanese 2) | 2.887/3.073 | 0.591 | 0.564 | 3.79 × 10⁻³ | 1.11 (1.04-1.20) | |
| Third set (Japanese 3) | 3.558/2.308 | 0.588 | 0.563 | 0.0105 | 1.10 (1.02-1.19) | |
| All Japanese^{b} | 10.782/8.451 | 0.592 | 0.562 | 3.66 × 10⁻⁹ | 1.13 (1.09-1.18) | 0.474 |
| East Asian without Japanese^{b} | 4.151/4.055 | 0.669^{c} | 0.654^{c} | 0.0126 | 1.09 (1.02-1.16) | 0.460 |
| All east Asian^{b} | 14.933/12.506 | 0.614 | 0.592 | 2.61 × 10⁻¹⁰ | 1.12 (1.08-1.16) | 0.533 |
| All European^{b} | 6.798/7.871 | 0.590^{d} | 0.566^{d} | 6.36 × 10⁻⁶ | 1.10 (1.05-1.15) | 0.347 |
| All populations^{b} | 21.731/20.377 | 0.606 | 0.582 | 8.78 × 10⁻¹⁴ | 1.11 (1.08-1.14) | 0.622 |
| rs1436953 | | | | | | |
| First set (Japanese 1) | 4.337/3.071^{a} | 0.624 | 0.585 | 2.32 × 10⁻⁶ | 1.17 (1.10-1.26) | |
| Second net (Japanese 2) | 2.883/3.073 | 0.613 | 0.593 | 0.0242 | 1 09 (1.01-1.17) | |
| Third set (Japanese 3) | 3.569/2.292 | 0.612 | 0.588 | 0.0102 | 1.11 (1.02-1.19) | |
| All Japanese^{b} | 10.789/8.436 | 0.617 | 0.589 | 2.19 × 10⁻⁶ | 1.13 (1.08-1.17) | 0.284 |
| East Asian without Japanese^{b} | 2.760/2.570 | 0.680^{c} | 0.668^{c} | 0.0978 | 1.07 (0.99-1.16) | 0.473 |
| All east Asian^{b} | 13.549/11.006 | 0.630 | 0.607 | 9.46 × 10⁻⁹ | 1.11 (1.07-1.16) | 0.387 |
| All European^{b} | 6,786/7.927 | 0.436^{d} | 0.420^{d} | 0.0162 | 1.06 (1.01-1.11) | 0.488 |
| All populations^{b} | 20.335/18,933 | 0.565 | 0.529 | 2.09 × 10⁻⁹ | 1.09 (1.06-1.12) | 0.280 |

^{a}In this analysis, subjects with T2D who were registered from [symbol] University of Medical Science or from [symbol] University were excluded to completely eliminate the possibility of subject overlap between the Japanese 1 and Japanese 3 populations.
^{b}Combined analysis with the Mantel-Haenszel test using a fixed effect model; the weighted means of the risk allele frequencies are presented.
^{c}The weighted means for the risk allele frequencies for three east Asian populations (Singaporean Han Chinese/Hong Kong Han Chinese/Korean populations): rs7172432, T2D 0.686/0.699/0.569, CN 0.658/0.688/0.563: rs 1436953, T2D 0.715/-/0.585, CN 0697/-/0.580. Singaporean Han Chinese population, n = 2.010 T2D cases and 1.945 CN cases; Hong Kong Han Chinese population, n = 1,416 T2D cases and 1, [symbol] 77 CN cases: Korean population, n = 758 T2D Cases and 632 CN cases.
^{d}The weighted means for the risk allele frequencies for two European populations (Danish and French populations): rs7172432, T2D 0.587/0.594, CN 0. [symbol] 59/0.578: rs1436953. T2D 0.428/0.444, CN 0.410/ 0.434. Danish population, n = 3,692 T2D cases and 5.046 CN cases; French population, n = 3,288 T2D cases and 3,5 [symbol] 9 CN cases. [symbol]
indicates data missing or illegible when filed

UBE2E2, located at 3p24.2, encodes the ubiquitin-conjugating enzyme E2E2 (Cytogenet. Cell Genet. 78, 107-111 (1997)), which is reported to be expressed in human pancreas, liver, muscle and adipose tissue, as well as in a cultured insulin-secreting cell line. It has been reported that an ubiquitin-proteasome system plays a pivotal role in maintaining normal insulin biosynthesis, secretion and signaling, especially under conditions that increase endoplasmic reticulum stress in pancreatic β cells (Am. J. Physiol. Endocrinol. Metab. 296, E1-E10 (2009)). Several reports showed that proteasome inhibition by pharmacological inhibitors reduced proinsulin biosynthesis (J. Biol. Chem. 280, 15727-15734 (2005)), the activity of molecules involved in insulin secretion (J. Biol. Chem. 281, 13015-13020 (2006)) and glucose-stimulated insulin secretion (Diabetes 55, 1223-1231 (2006) and Diabetologia 28, 412-419 (1985)), whereas other investigators reported that proteasome inhibitors enhanced acute glucose-induced insulin secretion in isolated rat islets (Gene 342, 85-95 (2004)). These reports both suggested that the ubiquitin-proteasome system plays important roles in insulin secretion. Among the 872 control subjects in stage 3 (FIG. 1) whose fasting plasma glucose and insulin levels were available, subjects having the risk allele rs7612463 (CC+CA; n=846) showed a significantly lower homeostasis model assessment of β-cell function (HOMA-β) (Diabetologia 28, 412-419 (1985)) than those without the risk allele (AA; n=26) (P=0.0163; 73.7±36.1 compared to 90.8±39.0), suggesting a role for this variant in reducing insulin secretion.

We also examined association of SNPs within 400 kb around the C2CD4A-C2CD4B locus and found that the susceptibility locus in this region was likely localized between C2CD4A and C2CD4B (data not shown). C2CD4A-C2CD4B (encoding C2 calcium-dependent domain containing 4), also known as NLF1-2 (encoding nuclear localized factor) or FAM148A-B (encoding family with sequence similarity 148), are located at 15q22.2 and encode nuclear factors with a role in regulating genes that control cellular architecture (Gene 342, 85-95 (2004)). Functional roles of C2CD4A-C2CD4B encoded proteins, however, are not well characterized, and evidence of a role for C2CD4A-C2CD4B in conferring susceptibility to T2D has previously been lacking, although expression of these genes was reported in human pancreas, liver, muscle and adipose tissue, as well as in a cultured insulin-secreting cell line, and expression of C2CD4A-C2CD4B has been shown to be increased by treatment with pro-inflammatory cytokines (Gene 342, 85-95 (2004).).

### INDUSTRIAL APPLICABILITY

According to the method of the present invention, type II diabetes can be detected, which is useful in the fields of diagnosis and the like. Further, according to the screening method of the present invention, novel medicaments for type II diabetes can be obtained, which is useful in medical fields and the like.

While the invention has been described in detail with reference to preferred embodiments thereof, it will be apparent to one skilled in the art that various changes can be made, and equivalents employed, without departing from the scope of the invention. Each of the aforementioned documents as well as U.S. 61/379,489 is incorporated by reference herein in its entirety.

1. A method of detecting type II diabetes, comprising:
analyzing a single-nucleotide polymorphism in the UBE2E2 locus or C2CD4A-C2CD4B locus, and
detecting type II diabetes based on the result of the analysis.

2. The method according to claim 1, wherein a single-nucleotide polymorphism in the UBE2E2 locus is a polymorphism of a nucleotide corresponding to the nucleotide at position 61 of SEQ ID NO: 1, SEQ ID NO: 2, or SEQ ID NO: 3, or of a nucleotide in linkage disequilibrium with the nucleotide.

3. The method according to claim 1, wherein a single-nucleotide polymorphism in the C2CD4A-C2CD4B locus is a polymorphism of a nucleotide corresponding to the nucleotide at position 61 of SEQ ID NO: 4, SEQ ID NO: 5, or SEQ ID NO: 6, or of a nucleotide in linkage disequilibrium with the nucleotide.

4. A probe for detecting type II diabetes, which comprises a sequence of 10 or more consecutive nucleotides in SEQ ID NO: 1, 2, 3, 4, 5, or 6 including the nucleotide at position 61, or a complementary sequence thereof.

5. A primer for detecting type II diabetes, which is capable of amplifying a region comprising the nucleotide at position 61 of SEQ ID NO: 1, 2, 3, 4, 5, or 6.
